SEQUENCE LISTING (9 sequences)

SEQ ID NO: 1 - DNA, 20 bp, Artificial Sequence
  primer
gatgagcaggtccgaggtta

SEQ ID NO: 2 - DNA, 20 bp, Artificial Sequence
  primer
tcttccaaggcagctttcat

SEQ ID NO: 3 - DNA, 23 bp, Artificial Sequence
  probe
ctgcggcgcacccttcagggtct

SEQ ID NO: 4 - DNA, 25 bp, Artificial Sequence
  primer
taatgagagtgagggaatacctttg

SEQ ID NO: 5 - DNA, 25 bp, Artificial Sequence
  primer
aggcaagttttcatcaaatagttca

SEQ ID NO: 6 - DNA, 22 bp, Artificial Sequence
  probe
ggcgtgtgtcctttggtgggca

SEQ ID NO: 7 - DNA, 27 bp, Artificial Sequence
  primer
cacagtgaatcttggttgtaaacttga

SEQ ID NO: 8 - DNA, 22 bp, Artificial Sequence
  primer
aaaccgcttgggattatattcg

SEQ ID NO: 9 - DNA, 24 bp, Artificial Sequence
  probe
aagaccaatgcacttcgtgcccga

(19) United States
(12) Patent Application Publication
(10) Pub. No.: US 2012/0244531 A1
(43) Pub. Date: Sep. 27, 2012

(54) METHOD FOR PROVIDING INFORMATION FOR DIAGNOSING CANCER USING QUANTITATIVE REAL-TIME PCR AND KIT FOR DIAGNOSING CANCER FOR THE SAME

(75) Inventors: Hye-Young LEE, Wonju (KR); Seung-II Kim, Seoul (KR); Sang-Jung Park, Wonju (KR); Tae-Ue Kim, Wonju (KR); Yoon-Jung Cho, Busan (KR); Hyun-Ju Han, Seoul (KR)

(73) Assignee: M&D, INC, Wonju (KR)

(21) Appl. No.: 13/362,490

(22) Filed: Jan. 31, 2012

(30) Foreign Application Priority Data
Mar. 25, 2011 (KR) 10-2011-0049725

(51) Int. Cl. C12Q 1/68

(52) U.S. Cl. 435/6.11

(57) ABSTRACT

There is provided a method for providing information for diagnosing cancer using Real-Time RT-PCR, and a kit for diagnosing cancer for the method.

### CROSS-REFERENCE TO RELATED APPLICATIONS

This application claims the priority of Korean Patent Application No. 10-2011-0049725 filed on May 25, 2011, in the Korean Intellectual Property Office, the disclosure of which is incorporated herein by reference.

### BACKGROUND OF THE INVENTION

1. Field of the Invention

The present invention relates to a method for providing information for diagnosing cancer using a quantitative Real-Time PCR and a kit for diagnosing cancer for the same.

2. Description of the Related Art

Our country currently has the highest death rate from cancer so that cancer is an important disease; and cancer development is increasing every year and our country reported a cancer rate of at least 300 per 100,000 in 2008 (Jemal, A.; Siegel, R.; Xu, J.; Ward, E., Cancer statistics, 2010. CA Cancer J Clin 2010, 60, (5), 277-300).

In the case of the early stage of cancer, most malignancies can be cured by a simple operation or a drug treatment, but if cancer has spread to other organs, the cancer is difficult to treat, and also the prognosis and the survival rate after 5 years of patient are decreasing. In the case of the metastatic cancer as mentioned above, a cancer cell presented in the initial solid cancer comes out of the original lesion to settle in other organs through a lymph node, a blood stream, or a born-marrow thereby inducing the metastatic cancer. The cancer cell presented in the blood stream as mentioned above is called a circulating tumor cell (Ross, J. S.; Slodkowska, E. A., Circulating and disseminated tumor cells in the management of breast cancer. Am J Clin Pathol 2009, 132, (2), 237-45).

The circulating tumor cell is called a minority of tumor cells that come out of the primary tumor tissue and then get around through the blood flow, and it may get around in the blood and then may cause metastatic cancer at a secondary region. Therefore, the diagnosis of the circulating tumor cell may be effectively used for detecting as to whether the cancer has expanded for a patient with early stage cancer. Since it is generally known that the progress of the patient with the metastatic cancer can react badly to the treatment and the prognosis after the operation is bad, the diagnosis of the circulating tumor cell is being used as a good marker for detecting the progress or the prognosis of the patient because it can help to measure as to whether the cancer of the patient can be progressed to the metastatic cancer (Meng, S.; Tripathy, D.; Frenkel, E. P.; Shete, S.; Naftalis, E. Z.; Huth, J. F.; Beitsch, P. D.; Leitch, M.; Hoover, S.; Euhus, D.; Haley, B.; Morrison, L.; Fleming, T. P.; Herlyn, D.; Terstappen, L. W.; Fehm, T.; Tucker, T. F.; Lane, N.; Wang, J.; Uhr, J. W., Circulating tumor cells in patients with breast cancer dormancy. Clin Cancer Res 2004, 10, (24), 8152-62; Pachmann, K., Longtime recirculating tumor cells in breast cancer patients. Clin Cancer Res 2005, 11, (15), 5657; author reply 5657-8).

In addition, the cancer cell has a significantly fast differentiation speed of cell unlike a general cell. Therefore, if it is expressed by using the marker used for the cell differentiation that the differentiation speed is faster, it can be found that there are certain diseases and disorders.

Currently, a method for a diagnosis of metastatic cancer is being made by detecting as to whether a cancer cell is presented using a detecting method by an invasive lymph node, and an image equipment, such as MRI and PET. However, it has a limitation that the detecting method as mentioned above can use in the case of the metastatic cancer. However, the prognosis management of the cancer patient may be carefully made by diagnosing the patient that has a possibility to expand among the patients with early stage cancer that have not expanded by testing as to whether the circulating tumor cell is presented.

An antigen-antibody detecting method using the difference between the surface antigens of cancer cells and blood cells is very popular. The surface of cancer cells has the epithelium antigen, as distinct from the blood cells. The antigens as mentioned above are presented on the epithelium cell, but are not presented at the interior wall of blood vessel and the blood cells. Especially, it is known that Cytokeratin 19 among the antigens as mentioned above is expressed on breast cancer, bladder cancer, cervical cancer, colorectal cancer, lung cancer, pancreatic cancer, stomach cancer, and the like (Karantza, V., Keratins in health and cancer: more than mere epithelial cell markers. Oncogene 2011, 30, (2), 127-38). Therefore, a method for diagnosing the circulating tumor cell is currently being used using Cytokeratin 19 antigen. As a method for diagnosing that is currently being used, CellSearch from Veridex Inc. is the most commonly used, in which the method is to test as to whether the circulating tumor cell is presented using Pan-Cytokeratin antibody after distinguishing the cells that are negative to CD45 and positive to EpCAM through Antigen-antibody binding reaction using a fluorescent-marked antibody (Allard, W. J.; Matera, J.; Miller, M. C.; Repollet, M.; Connelly, M. C.; Rao, C.; Tibbe, A. G.; Uhr, J. W.; Terstappen, L. W., Tumor cells circulate in the peripheral blood of all major carcinomas but not in healthy subjects or patients with nonmalignant diseases. Clin Cancer Res 2004, 10, (20), 6897-904). However, the technique as mentioned above is currently not introduced in our country, and also it has a limitation that it requires expensive equipment. In addition, a method for diagnosing the circulating tumor cell based on RT-PCR that is now developed has limitations because it detects the bands of amplified fragments using an electrophoresis, as follows: the specificity is low; the experiment process is complicated, and the quantitative results are difficult to obtain. Therefore, the present inventors invented a new method for diagnosing the circulating tumor cell based on Real-time RT-PCR that can obtain a simple and a quantitative result by targeting mRAN not Antigen-antibody reaction in order to replace the above mentioned technique.

### SUMMARY OF THE INVENTION

The present invention is for solving the above limitations and invented for the above needs, and an object of the present invention is to provide a method for diagnosing a circulating tumor cell based on Real-Time RT-PCR.

Another of the present invention is to provide a kit for diagnosing the circulating tumor cell.

In order to achieve the above objects, the present invention provides a method for providing information for diagnosing cancer, comprising: a) isolating full-length RNA from cells (all cells except a red blood cell) obtained from blood of a patient suspicious for cancer; b) synthesizing cDNA from the isolated full-length RNA; c) performing Real-Time PCR with the synthesized cDNA using at least one primer pair and probe selected from the group consisting of a primer pair and a probe that can amplify Cytokeratin 19, a primer pair and a probe that can amplify Ki67, and a primer pair and a probe that can amplify TBP; and d) comparing the amplified amount of the above step with the amplified amount of the normal.

A method for isolating full-length RNA (Total RNA) and a method for synthesizing cDNA from the isolated full-length RNA that are generally used can be performed through the known method, and the detailed description about the process is disclosed in Joseph Sambrook et al., Molecular Cloning, A Laboratory Manual, Cold Spring Harbor Laboratory Press, Cold Spring Harbor, N.Y. (2001); and Noonan, K. F., and the like, which may be incorporated in the present invention as a reference.

The primer of the present invention can be chemically synthesized using a phosphoramidite solid support method, or other known methods. The nucleic acid sequence may be also modified by using many ways that are known in the prior art. The non-limited example of the modifications may be a methylation, “a capping,” a substitution with at least one homologue of natural nucleotide, and the modification between nucleotides, for example, the modification to a non-charged linker (For example: methyl phosphonate, phosphotriester, phosphoroamidate, carbamate, and the like) or a charged linker (For example: phosphothioate, phosphorodithioate, and the like). Nucleic acid may include at least one additionally covalent bonded residue, for example, protein (For example: nuclease, toxin, antibody, signal peptide, poly-L-lysine, and the like), an intercalating agent (For example: acridine, psoralens, and the like), a chelating agent (For example: metal, radioactive metal, iron, oxidative metal, and the like), and an alkylation agent. The nucleic acid sequence of the present invention may be also modified with the marker that can directly or indirectly provide a detectable signal. Example of the marker may include a radioactive isotope, fluorescence molecular, a biotin, and the like.

For the method according to the present invention, the amplified target sequence (Gene, such as Cytokeratin 19, Ki67, and the like) may be marked with a detectable marker material. For one embodiment, the marker material may be the material that emits a fluorescence, a phosphorescence, a chemiluminescence, and a radioactivity, but limited thereto. Preferably, the marker material may be fluorescein, phycoerythrin, rhodamine, lissamine Cy-5 or Cy-3. The marker material may be marked with a detectable fluorescent marker material by performing Real-Time RT-PCR through marking Cy-5 or Cy-3 to 5′-end and/or 3′-end of primer when amplifying a target sequence.

In addition, the marking using the radioactive material may be achieved by marking the amplified fragment with a radioactivity through incorporating the radioactivity to the amplified fragment while synthesizing the amplified fragment by adding a radioactive isotope, such as ³²P or ³⁵S to PCR reaction solution when performing Real-Time RT-PCR. At least one oligonucleotide primer set that is used for amplifying the target sequence may be used.

The marking may be performed through various methods that are typically performed in the art, such as, a nick translation method, a random priming method [Multiprime DNA labelling systems. booklet, “Amersham” (1989)], and a kination method [Maxam & Gilbert, Methods in Enzymology, 65:499 (1986)]. The marker provides a detectable signal by fluorescence, a radioactivity, a chromophore measurement, a weight measurement, X-ray diffraction or absorption, magnetism, an enzymatic activity, amass analysis, a binding affinity, a hybridization high frequency, and a nano crystal.

According to one aspect of the present invention, the present invention measures the level of expression in the level of mRNA through RT-PCR. To achieve this, new primer pair and fluorescent-marked probe that are specifically bonded to Cytokeratin 19, Ki67, and TBP genes are required; the primer and probe specified with the specific base sequence for the present invention can be used, but not limited thereto; if they provide the detectable signal by specifically binding to those genes and can perform Real-Time RT-PCR, they can be used without limit. In the above sentence, FAN and BHQ1 mean a fluorescent dye.

Real-Time RT-PCR method applied for the present invention can be performed through the known process that is typically used in the art.

The measuring of mRNA expression level can be used without limit if it can measure a general mRNA expression level, and according to a type of used probe markers, it can be performed through a radioactivity measurement, a fluorescence measurement, or a phosphorescence measurement, but not limited thereto. As one of methods for detecting an amplified fragment, a fluorescence measuring method can be performed as follows: Cy-5 or Cy-3 is marked to 5′-end of primer and then Real-Time RT-PCR is performed with the marked primer to mark a fluorescent marker material to a target sequence; and then the marked fluorecence can be measured using a fluorometer. In addition, the radioactivity measuring method can be performed as follows: when performing Real-Time RT-PCR, an amplified fragment is marked with a radioactivity isotope, such as ³²P or ³⁵S, and the like by adding the radioactivity isotope to PCR reaction solution, and then the radioactivity can be measured using a radioactivity measuring instrument, such as Geiger counter or

### Liquid Scintillation Counter.

According to one preferable embodiment of the present invention, a fluorescent-marked probe is attached to PCR product amplified through the Real-Time PCR to fluoresce a specific wavelength; the expression levels of mRNAs of genes according to the present invention are measured in real time with a fluorometer of Real-Time PCR apparatus at the same-time with the above amplification; and then the measured values are calculated and visualized through PC so that a tester can easily confirm the expression levels.

According to one embodiment of the present invention, the comparing of the above amplified amount with the amplified amount of the normal is preferably performed by a standard or Cut-Off value, and for the Cut-Off value, Ct value (Threshold Cycle) is more preferably 34.0, but is not limited thereto.

According to another embodiment of the present invention, preferably, the primer pair has the base sequences that are disclosed as Sequence Nos. 1 & 2 and the probe has the base sequence that is disclosed as Sequence No. 3, in which the primer pair and the probe can amplify Cytokeratin 19.

Preferably, the primer pair has the base sequences that are disclosed as Sequence Nos. 4 & 5 and the probe has the base sequence that is disclosed as Sequence No. 6, in which the primer pair and the probe can amplify Ki67.

Preferably, the primer pair has the base sequences that are disclosed as Sequence Nos. 7 & 8, and the probe has the base sequence that is disclosed as Sequence No. 9, in which the primer pair and the probe can amplify TBP.

However, the above primers and the probes are not limited thereto.

Additionally, the present invention provides a primer pair and a probe for diagnosing cancer, including at least one primer pair and probe selected from the group consisting of: a primer pair having the base sequences that are disclosed as Sequence Nos. 1 & 2 and the probe having the base sequence that is disclosed as Sequence No. 3, which can amplify Cytokeratin 19; a primer pair having the base sequences that are disclosed as Sequence Nos. 4 & 5 and the probe having the base sequence that is disclosed as Sequence No. 6, which can amplify Ki67; and a primer pair having the base sequences that are disclosed as Sequence Nos. 7 & 8 and the probe having the base sequence that is disclosed as Sequence No. 9, which can amplify TBP.

Additionally, the present invention provides a composition for diagnosing cancer, including the primer pair and probe according to the present invention.

According to one embodiment of the present invention, the cancer may be preferably breast cancer, bladder cancer, cervical cancer, colorectal cancer, lung cancer, pancreatic cancer, stomach cancer, ovarian cancer, blood cancer, liver cancer, prostate cancer, or head and neck cancer, but is not limited thereto.

Additionally, the present invention provides a kit for diagnosing cancer, including the composition according to the present invention.

According to another aspect of the present invention, the kit for diagnosing may be a kit for diagnosing cancer, in which the kit for diagnosing includes essential ingredients that need for performing RT-PCR. RT-PCR kit may include each specific primer pair to the genes according to the present invention. The primer is a nucleotide having a specific sequence to nucleic acid sequence of each marker gene; the length of the primer may be about 7 by to 50 bp, more preferably about 10 by to 30 bp; and more preferably the primer may include new primer pair that is expressed as Sequence Nos. 1 & 2 and a fluorescent-marked probe that is expressed as Sequence No. 3; new primer pair that is expressed as Sequence Nos. 4 & 5 and a fluorescent-marked probe that is expressed as Sequence No. 6; and/or new primer pair that is expressed as Sequence Nos. 7 & 8 and a fluorescent-marked probe that is expressed as Sequence No. 9.

And also, RT-PCR kit may include a test tube or other proper container, a reaction buffer (pH and magnesium concentration are varied), deoxynucleotide (dNTPs), Taq-polymerase, and enzyme, such as a reverse transcriptase, DNAse, RNAse inhibitor, DEPC-water, a sterilized water, and the like.

The term, “Method for providing information for diagnosing cancer” in the present invention is a preliminary stage for diagnosing to provide objective basic information required for diagnosing cancer, but not include a clinical judgment and opinion by a doctor.

The term, “Primer” indicates a nucleic acid sequence having a short free 3′ hydroxyl group; can form a complementary template and base pair; and indicates a short nucleic acid sequence having the function of starting point in order for the template strand transcription. The primer can start DNA synthesis under presence of different four nucleoside triphosphates and the reagent for a polymerization (that is, DNA polymerase or reverse transcriptase) in a proper buffer solution and at a proper temperature. The primer according to the present invention is sense and anti-sense nucleic acid having 7 to 50 nucleotide sequences, which is each marker gene-specific primer. The primer may be incorporated with an additional feature that cannot change a basic property of primer that acts as a starting point of DNA synthesis.

The term, “Probe” is a single chain nucleic acid molecular, and includes a complementary sequence to a target nucleic acid sequence.

The term, “Real-Time RT-PCR” is a molecular-biological polymerization method to quantitatively detect the signal generated at the marker of the target probe, in which the target is amplified using a target probe including a target primer and marker using cDNA as a template, and the cDNA is produced after Reverse-transcription of RNA with a complementary DNA (cDNA) using a reverse transcriptase.

Hereinafter, the present invention will be described.

The present inventors provides a test method that can confirm the potential metastatic cancer patients by testing as to whether circulating tumor cells are presented after collecting one-tube of blood from cancer patient, in which the metastatic cancer cannot be detected with the existed image equipment. Especially, there are advantages that since the method uses a method for amplifying a gene using mRNAs of Ki67 that is a cell division marker and Cytoketatin 19 that is an epithelial antigen, it can detect the unseeable amount and since it does not use Antigen-Antibody reaction, it is very cheap method.

Hereinafter, the present invention will be described in more detail.

Confirmation as to Whether Cytokeratin 19 and Ki67 are Expressed in Each of Cell Lines

It was confirmed as to whether Cytokeratin 19 and Ki67 were expressed using cell lines that are corresponded to each of cancer cells. The cell lines used included three types of breast cancer-cell line (MCF7, SKBR3, MDA-MB 231), lung cancer-cell line (A549), cervical cancer-cell line (HeLa), and ovarian cancer-cell line (SKOV3), and also human monocyte-cell line (THP-1) was used as a negative control.

The cell number of each cell line was maintained at 100,000 to confirm the expression aspect of Cytokeratin 19 per cell line. As a result, it could be found that Cytokeratin 19 was highly expressed in the breast cancer-cell line and lung cancer-cell line and was not expressed in the ovarian cancer-cell line and monocyte-cell line. It could be also found that Ki67 was expressed in all of cancer cells.

TABLE 1

| | Clinical Specimen | Cytokeratin 19 | |
|---|---|---|---|
| Well | Name | (Ct value) | Note |
| A | MCF7 | 20.62 | Breast Cancer |
| B | SKBR3 | 21.30 | Breast Cancer |
| C | MDA-MB 231 | 24.65 | Breast Cancer |
| D | A549 | 27.17 | Lung Cancer |
| E | HeLa | 34.66 | Cervical Cancer |
| F | SKOV3 | Undetermined | Ovarian Cancer |
| G | THP-1 | Undetermined | Monocyte |
| H | Blank control | Undetermined | |

Table 1 shown the expression aspects of Cytokeratin 19 per cell lines.

TABLE 2

| | Clinical Specimen | Ki67 | |
|---|---|---|---|
| Well | Name | (Ct value) | Note |
| A | MCF7 | 23.49 | Breast Cancer |
| B | SKBR3 | 23.65 | Breast Cancer |
| C | MDA-MB 231 | 22.91 | Breast Cancer |
| D | A549 | 26.64 | Lung Cancer |
| E | HeLa | 25.03 | Cervical Cancer |
| F | SKOV3 | 31.82 | Ovarian Cancer |
| G | THP-1 | 23.00 | Monocyte |
| H | Blank control | Undetermined | |

Table 2 shown the expression aspects of Ki67 per cell lines.

Confirmation as to Whether Ki67 is Expressed, Cut-Off Value Set-Up, and Sensitivity of Cytokeratin 19 Expression

10 ml of blood was collected from healthy human without cancer, a model of circulating tumor cell was intentionally prepared by mixing from 10⁵ to 1 cell of MCF7 that was the breast cancer-cell line with the above collected blood by stages, and then the sensitivity was confirmed. In addition, Cut-Off value was experimentally set-up using the blood of healthy human without cancer. As a result, 10 per 10 ml in the circulating tumor cell intentionally prepared could be detected, and also Cut-Off value of Ct value could be set-up as 34.0.

In the case of Ki67, after TBP (TATA BOX BINDING Protein) and Ki67 were amplified, respectively, Ct values were measured, and then the expression aspects were confirmed based on the normal 4. The expression aspects were confirmed from 1.0 to 3.27.

TBP in the present invention was used as Internal control. In the case of Ki67, TBP was added at all times in order to set the standard unlike Cytokeratin 19 (As to whether Cytokeratin 19 is expressed can be confirmed by detecting a relative cell number through preparing a quantitative curve using the circulating tumor cell model as a standard) and TBP and ki67 were added at all times based on DNA with the same concentration with Ct value of TBP of the normal in order to compare the expression amount of the normal. When at least 10 times was expressed after the standard of TBP was set-up to 1, it was tested the positive.

TABLE 3

| Clinical | Cytokeratin 19 | |
|---|---|---|
| Specimen Name | (Ct value) | Relative Cell No. |
| Blood + MCF7 10⁵ | 20.6 | 100000.0 |
| Blood + MCF7 10⁴ | 24.0 | 10000.0 |
| Blood + MCF7 10³ | 28.2 | 1000.0 |
| Blood + MCF7 10² | 31.4 | 100.0 |
| Blood + MCF7 10 | 32.0 | 10.0 |
| Blood + MCF7 1 | 34.0 | 1.0 |
| Normal 1 | 34.8 | |
| Normal 2 | 35.1 | |
| Normal 3 | 37.2 | |
| Normal 4 | 35.9 | |

Table 3 shown the sensitivities of circulating tumor cells models.

TABLE 4

| Clinical Specimen | TBP | Ki67 | |
|---|---|---|---|
| Name | (Ct value) | (Ct value) | Expression |
| Normal 1 | 27.10 | 31.6 | 1.77 |
| Normal 2 | 28.71 | 31.0 | 3.29 |
| Normal 3 | 27.80 | 31.9 | 2.77 |
| Normal 4 | 27.0 | 32.6 | 1.00 |

Table 4 shown Ki67 expressions of the normal.

Confirmation of Ki67 and Cytokeratin 19 in the Blood of the Patient with Metastatic Breast Cancer

It was confirmed as to whether Cytokeratin 19 was expressed after receiving the blood of the patients with the metastatic breast cancer from Sinchon Severance Hospital. After a quantitative curve was prepared using the arbitrary circulating tumor cell model used for the above test as a standard, a relative cell number was confirmed. As a result, there was one patient, in which at least 10 of the circulating tumor cell among total 6 patients was detected, and there was two patients having not more than Cut-off value but not at least 10 of the circulating tumor cell.

In addition, it could be found that Ki67 was highly expressed in the patient group that shown high Cytokeratin 19 expression as compared with the normal. Considering maximum 3.29 of expression rate of the normal, it could be found that Ki67 was expressed at a high rate in the patient group 4 and patient group 6, respectively. Therefore, when detecting using two markers at the same time, the positive ratio can be more increased so that it could be more easily used for observing the cancer patients who could develop the metastatic cancer.

TABLE 5

| Clinical Specimen | Cytokeratin 19 | |
|---|---|---|
| Name | (Ct value) | Relative Cell No. |
| Blood + MCF7 10⁵ | 20.6 | 100000.0 |
| Blood + MCF7 10⁴ | 24.0 | 10000.0 |
| Blood + MCF7 10³ | 28.2 | 1000.0 |
| Blood + MCF7 10² | 31.4 | 100.0 |
| Blood + MCF7 10 | 32.0 | 10.0 |
| Blood + MCF7 1 | 34.0 | 1.0 |
| Patient Group 1 | 34.4 | 1.9 |
| Patient Group 2 | 34.4 | 1.8 |
| Patient Group 3 | 33.8 | 3.0 |
| Patient Group 4 | 30.7 | 43.9 |
| Patient Group 5 | 35.4 | 0.8 |
| Patient Group 6 | 33.7 | 3.2 |

Table 5 is a table for detecting the circulating tumor cell in the patients with metastatic breast cancer.

TABLE 6

| Clinical Specimen | TBP | Ki67 | Ki67 |
|---|---|---|---|
| Name | (Ct value) | (Ct value) | Expression rate |
| Patent Group 1 | 26.9 | 30.5 | 4.08 |
| Patent Group 2 | 26.0 | 29.2 | 5.47 |
| Patent Group 3 | 27.0 | 31.5 | 2.0 |
| Patent Group 4 | 29.6 | 31.2 | 16.0 |
| Patent Group 5 | 27.5 | 31.9 | 2.3 |
| Patent Group 6 | 29.6 | 31.0 | 18.4 |

Table 6 shown the expression aspects of Ki67 in the patient groups.

Comparison with Existed Known RT-PCR Method

In order to compare with a method for detecting the circulating tumor cell using the existed known RT-PCR method, the comparison experiment with Primer Set that was already used in other patent was performed. As a result, in the case of the existed known primer set, it could be found that the band with the same size as the band of Cytokeratin 19 was presented in the normal clinical specimen 4, but when using Real-Time PCR method used for the present invention, it could be not confirmed as to whether it was expressed, so that it could be found that the new developed primer and probe set had high specificity as compared with the existed known RT-PCR. In addition, the step for confirming the band using an electrophoresis was not required so that it had an advantage such that the result could be easily found.

The present invention can confirm the potential metastatic cancer patients by testing as to whether circulating tumor cells are presented after collecting one-tube of blood from cancer patient, in which the metastatic cancer cannot be detected with the existed image equipment. Especially, according to the present invention, there are advantages that since the method uses a method for amplifying a gene using mRNAs of Ki67 that is a cell division marker and Cytoketatin 19 that is an epithelial antigen, it can detect the unseeable amount, and since it does not use Antigen-Antibody reaction, it is very cheap method. Additionally, it could be found that the present invention had high specificity as compared with the existed known RT-PCR method, and also the result could be more easily confirmed because the step for confirming the band using an electrophoresis was not required.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a diagram showing as to whether Cytokeratin 19 is expressed per cell lines, in which A; MCF7, B; SKBR3, C; MDA-MB 231, D; A549, E; HeLa, F: SKOV3, G; THP-1, and H; Blank Control.

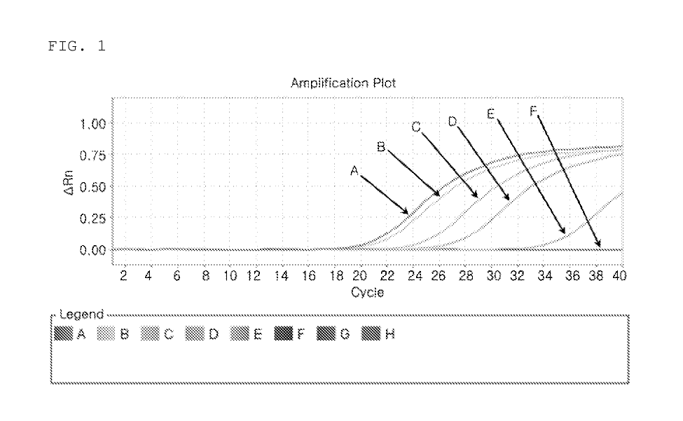

FIG. 2 is a diagram showing as to whether Ki67 is expressed per cell lines, in which A; MCF7, B; SKBR3, C; MDA-MB 231, D; A549, E; HeLa, F: SKOV3, G; THP-1, and H; Blank Control.

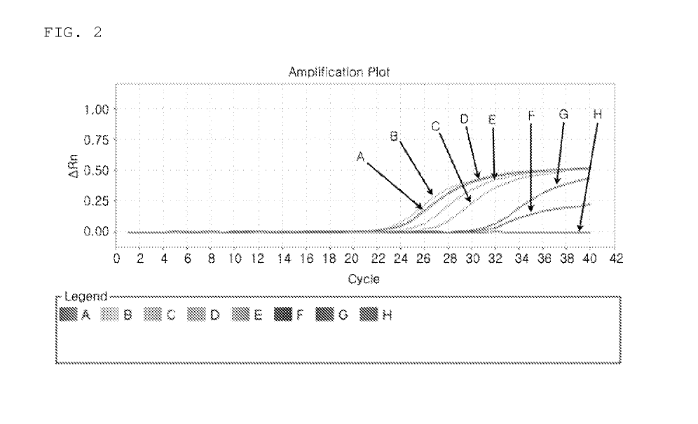

FIG. 3 is a diagram showing the detection of Cytokeratin in a metastatic breast cancer patient, in which Red Quadrangle; Standard Curve (Blood+MCF7 10⁵-1), and Blue Quadrangle; Patient Group.

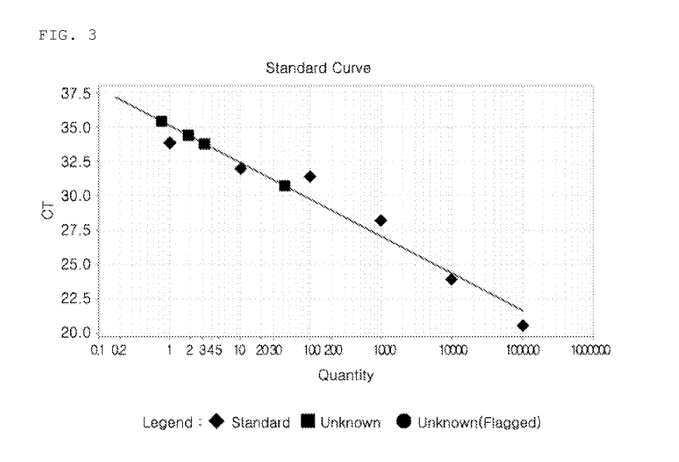

FIG. 4 is a diagram showing the confirmation of sensitivity and specificity of RT-PCR method to cytokeratin 19. MCF7 that is a breast cancer-cell was diluted from 10⁵ to 1 cell by stages with 10 ml of healthy person's blood, and then the experiment was performed. As a result, it could be detected to one cell. In addition, as a result for confirming the expression aspect in each cancer-cell line, it could be found that the cell was not detected in a monocyte and the band of Cytokeratin 19 could be confirmed in each cancer-cell line. \textcircled{1} THP-1 (Monocyte), C SKOV3 (Ovarian cancer-Cell line) \textcircled{3} HeLa (Cervical cancer-Cell line) \textcircled{4}˜\textcircled{6} Breast cancer-Cell line (MDA-MB 231, MCF7, SKBR3) \textcircled{7} Blank control. Additionally, in the case of Patient Group (P1-P6), the band of Cytokeratin 19 was confirmed in all of 6 clinical specimens and also the band was confirmed in one group of the normal (N1-N4).

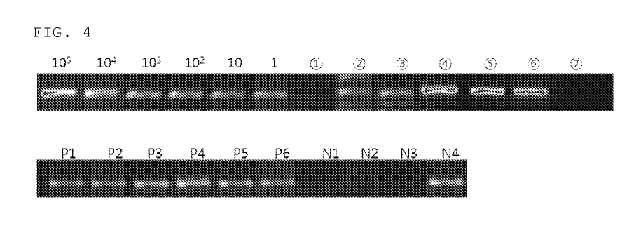

FIG. 5 is a diagram showing Primer positions of the existed method and the present invention, in which Red color indicates the position of primer used for the existed method (516-675), Blue color indicates the position of primer used for the present invention (1012-1107), and Green color indicates the position of probe used for the present invention (1037-1059).

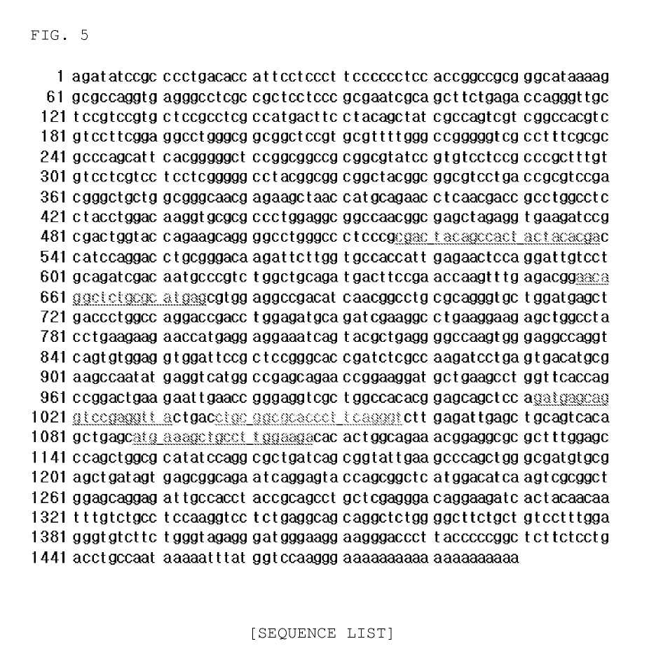

### DETAILED DESCRIPTION OF THE PREFERRED EMBODIMENT

Hereinafter, the present invention will be described in more detail through the non-limited Examples. However, Examples are only for illustrating the present invention and the range of the present invention is not limited to Example.

### Example 1

### Isolation of Cell from Patient's Blood

The blood was collected to EDTA tube from the vein of cancer patient. 5 ml of the blood that was first collected was discarded in order to prevent a contamination from an epithelial cell, and then 10 ml of the blood that was lately collected was used for the test method. An erythrocyte lysis process that was a first process should be started within 4 hours after collecting the blood in order to prevent mRNA damage from the patient's blood. In order to lysis an erythrocyte from the blood, 5 times volume of the erythrocyte lysis solution containing 154 mM of NH₄Cl, 9 mM of KHCO₃, and 0.1 mM of EDTA was added, vortexed, maintained for 10 minutes at room temperature, centrifuged at 400 g and 4° C., and then the supernatant was carefully discarded. In order to remove extra erythrocyte, 10 ml of RBC lysis buffer was added; maintained for 5 minutes in an ice; again centrifuged at 3000 rpm and 4° C. for 2 minutes; the supernatant was carefully discarded; 1 ml of PBS was added; the pellet was again floated; and then RNase A (100 ug/ml) was treated for 5 minutes in order to remove a free nucleic acid that was presented in the blood.

### Example 2

### Total RNA Isolation from Isolated Cell

The pellet that was again floated was centrifuged at 3000 rpm and 4° C. for 2 minutes; the supernatant was removed with pipetting; then 1 ml of Trizol reagent (Invitrogen) was added and then Total RNA was isolated according to the protocol of the manufacturing firm.

### Example 3

### cDNA Production from Isolated Total RNA and Real-Time PCR Performance

1) cDNA Synthesis

cDNA was synthesized by adding 2 ug of the isolated total RNA, 0.25 ug of random primer (Invitrogen), 250 uM of dNTP (Cosmo gene tech), 50 mM of Tris-HCl (pH 8.3), 75 mM of KCl, 3 mM of MgCl₂, 8 mM of DTT, and 200 units of MMLV reverse transcriptase polymerase (Invitrogen), adding DW treated with DEPC to be 20 ul of the final volume, mixing, and then reacting the synthesizing reaction solution at 25° C. for 10 minutes, at 37° C. for 50 minutes, and then at 70° C. for 15 minutes in a thermocycler (ABI).

2) Real-Time PCR Performance

For the reactant composition of Real-Time PCR, 25 mM of TAPS (pH 9.3, 25° C.), 50 mM of KCl, 2 mM of MgCl₂, 1 mM of 2-mercaptoethanol, 200 μM of each dNTP, 1 unit of Tag polymerase (TAKARA), 1 pmole of Forward primer, 1 pmole of Reverse primer, 1 pmole of probe, and 2 ul of synthesized cDNA were added to be 20 ul of the finial volume and then perform.

Each of primers and probes were as follows:

Primer and Probe for Cytokeratin 19 (Amplified
Fragment 96 bp)
Forward:
(Sequence No. 1)
5′GATGAGCAGGTCCGAGGTTA-3′
Reverse:
(Sequence No. 2)
5′TCTTCCAAGGCAGCTTTCAT-3′
Probe:
(Sequence No. 3)
5′FAM-CTGCGGCGCACCCTTCAGGGTCT-BHQ1-3′
Primer and Probe for Ki67
Forward:
(Sequence No. 4)
5′TAATGAGAGTGAGGGAATACCTTTG-3
Reverse:
(Sequence No. 5)
5′AGGCAAGTTTTCATCAAATAGTTCA-3
Probe:
(Sequence No. 6)
5′FAM-GGCGTGTGTCCTTTGGTGGGCA-BHQ1-3
Primer and Probe for TBP
Forward:
(Sequence No. 7)
5′CACAGTGAATCTTGGTTGTAAACTTGA-3
Reverse:
(Sequence No. 8)
5′AAACCGCTTGGGATTATATTC G-3
Probe:
(Sequence No. 9)
5′FAM-AAGACCAATGCACTTCGTGCCCGA-BHQ1-3

PCR reaction was performed using ABI 7500Fast (Applied Biosystem) as follows: performing one time at a denaturation temperature, 94° C. for 5 minutes; and then performing 40 times the cycle of a denaturation temperature, 95° C. for 30 seconds and an annealing temperature, 55° C. for 20 seconds, repeatedly. In addition, the fluorescence measurement steps were added after each of the annealing processes to measure the fluorescent value that was increased per each of cycles.

### Example 4

### Result Analysis

Each of experiment results was analyzed using 7500 Software v2.0.4 (Applied Biosystem). In the case of Cytokeratin 19, MCF7 that was a breast cancer-cell was diluted by stages using 10 ml of normal blood from 10⁵ to 1 cell and then drawn the relative quantitative curve so that the amount of relative circulating tumor cell could be measured using Ct value. In the case of Ki67, Ki67 expression amount was compared and quantitatively weighed based on the expression amount of Ki67 expressed in the normal blood for checking the expression rate. At this point, each of Ki67 expression amounts was compared based on the expression amount of TBP that was a house keeping gene.

While the present invention has been shown and described in connection with the exemplary embodiments, it will be apparent to those skilled in the art that modifications and variations can be made without departing from the spirit and scope of the invention as defined by the appended claims.

What is claimed is:

1. A method for providing information for diagnosing cancer, comprising:
a) isolating full-length RNA from cells obtained from blood of a patient suspicious for cancer;
b) synthesizing cDNA from the isolated full-length RNA;
c) performing Real-Time PCR with the synthesized cDNA using at least one primer pair and probe selected from the group consisting of a primer pair and probe that can amplify Cytokeratin 19, a primer pair and a probe that can amplify Ki67, and a primer pair and a probe that can amplify TBP; and
d) comparing the amount of the expression with the amount of the expression to the normal.

2. The method of claim 1, wherein the comparing of the amount of the expression with the amount of the expression to the normal is performed by a standard or Cut-Off value.

3. The method of claim 2, wherein Ct value (Threshold Cycle) to Cytokeratin 19 is 34.0 for the Cut-Off value.

4. The method of claim 1, wherein the primer pair that can amplify Cytokeratin 19 has the base sequences that are disclosed as Sequence Nos. 1 & 2 and the probe that can amplify Cytokeratin 19 has the base sequence that is disclosed as Sequence No. 3.

5. The method of claim 1, wherein the primer pair that can amplify Ki67 has the base sequences that are disclosed as Sequence Nos. 4 & 5 and the probe that can amplify Ki67 has the base sequence that is disclosed as Sequence No. 6.

6. The method of claim 1, wherein the primer pair that can amplify TBP has the base sequences that are disclosed as Sequence Nos. 7 & 8 and the probe that can amplify TBP has the base sequence that is disclosed as Sequence No. 9.

7. A primer pair and a probe for diagnosing cancer, comprising at least one primer pair and probe selected from the group consisting of:
a primer pair having the base sequences of Sequence Nos. 1 & 2 and a probe having the base sequence of Sequence No. 3, in which the primer pair and the probe can amplify Cytokeratin 19;
a primer pair having the base sequences of Sequence Nos. 4 & 5 and a probe having the base sequence of Sequence No. 6, in which the primer pair and the probe can amplify Ki67; and
a primer pair having the base sequences of Sequence Nos. 7 & 8 and a probe having the base sequence of Sequence No. 9, in which the primer pair and the probe can amplify TBP.

8. A composition for diagnosing cancer, comprising the primer pair and the probe of claim 7.

9. The composition of claim 8, wherein the cancer is breast cancer, bladder cancer, cervical cancer, colorectal cancer, lung cancer, pancreatic cancer, stomach cancer, ovarian cancer, blood cancer, liver cancer, prostate cancer, or head and neck cancer.

10. The composition of claim 8, wherein the cancer is metastatic cancer.

11. A kit for diagnosing cancer, comprising the composition of claim 8.
